(19) United States
(12) Patent Application Publication
(10) Pub. No.: US 2016/0287163 A1
(43) Pub. Date: Oct. 6, 2016

(54) DEVICES AND METHODS FOR LOCATING AND TREATING ESOPHAGEAL STRICTURES

(71) Applicant: BOSTON SCIENTIFIC SCIMED, INC., Maple Grove, MN (US)

(72) Inventors: JOHN B. GOLDEN, Norton, MA (US); PETER L. DAYTON, Brookline, MA (US); GARY TRENDEL, Hopkinton, MA (US); MEGAN HODSON, Marlborough, MA (US); MICHAEL POWERS, Pepperell, MA (US); MILFORD MCCRARY, Chapel Hill, NC (US); SARAH PROZELLER, Somerville, MA (US); YOUSEF AWWAD, Brighton, MA (US); JONATHAN ZOLL, Brookline, MA (US); ERIC M. SCHNEIDER, Cumberland, RI (US); BENEDICTA C. CHUMO, Framingham, MA (US); BRIANA J. MORETTI, Smithfield, RI (US); AMANDA L. SMITH, Cedar Rapids, IA (US); TARA A. JAROBSKI, North Oxford, MA (US)

(21) Appl. No.: 15/085,506

(22) Filed: Mar. 30, 2016

Related U.S. Application Data
(60) Provisional application No. 62/142,181, filed on Apr. 2, 2015.

(51) Int. Cl. A61B 5/00; A61M 29/02

(52) U.S. Cl. CPC A61B 5/4233; A61M 29/02; A61B 5/6853

(57) ABSTRACT

Methods and devices for locating and dilating a stricture are disclosed herein. In some embodiments, a device for locating strictures comprises an elongate member having a proximal end and a distal end. In at least some additional embodiments, a stopper is attached to the distal end of the elongate member. Additionally, in at least some examples, distance markings are disposed on the elongate member.

### CROSS-REFERENCE TO RELATED APPLICATIONS

This application claims priority under 35 U.S.C. §119 to U.S. Provisional Application Ser. No. 62/142,181, filed Apr. 2, 2015, the entirety of which is incorporated herein by reference.

### TECHNICAL FIELD

The disclosure is directed to devices and methods for treating esophageal strictures. More particularly, the disclosure is directed to devices and methods for locating and dilating esophageal strictures.

### BACKGROUND

Esophageal strictures are narrowing or tightening of the esophagus. These strictures can cause swallowing difficulties and may prevent people who suffer from such strictures from getting adequate nutrition. In some cases, these esophageal strictures may be treated in a hospital or outpatient setting using an endoscope and one or more inflatable balloons or bougies.

### BRIEF SUMMARY

The disclosure is directed to several alternative designs, materials and methods of manufacturing medical device structures and assemblies, and uses thereof. In one embodiment, a device for locating strictures comprises an elongate member having a proximal end and a distal end and a stopper attached to the distal end of the elongate member. In at least some additional embodiments, distance markings are disposed on the elongate member.

Alternatively, or additionally, in the above embodiment, the stopper comprises a balloon member.

Alternatively, or additionally, in any of the above embodiments, the stopper is cone shaped, and wherein the base of the cone shape is attached to the distal end of the elongate member.

Alternatively, or additionally, in any of the above embodiments, the stopper is shaped as a frustum of a cone, and wherein the base on the frustum of the cone shape is attached to the distal end of the elongate member.

Alternatively, or additionally, in any of the above embodiments, the stopper is umbrella shaped.

Alternatively, or additionally, in any of the above embodiments, the stopper includes a living hinge.

Alternatively, or additionally, in any of the above embodiments, the elongate member further includes a frangible portion.

Alternatively, or additionally, in any of the above embodiments, the stopper contains one or more frangible portions.

Alternatively, or additionally, any of the above embodiments may further comprise a marker adjustably disposed on the elongate member.

In another embodiment, a method of locating a stricture comprises inserting a stricture locating device into an esophagus of a patient. In at least some embodiments, the stricture locating device comprises an elongate member having a proximal end and a distal end, a stopper attached to the distal end of the elongate member, and distance markings disposed on the elongate member. The method may additionally comprise pushing the stopper past the stricture. In at least some embodiments, the method may further comprise retracting the stricture locating device until the stopper contacts the stricture.

Alternatively, or additionally, in the above embodiment, the stricture locating device further comprises a marker adjustably disposed on the elongate member, the method further comprising: adjusting the marker to contact the lips or teeth of the patient.

In another embodiment, a dilation device for insertion into an esophagus comprises an elongate member having a proximal end and a distal end wherein the elongate member has a tip member bent at an angle relative to the elongate member disposed at the distal end of the elongate member. In at least some of these embodiments, the dilation device may further comprise a reservoir connected to the proximal end of the elongate member and a valve disposed between the proximal end of the elongate member and the reservoir. Additionally, in some embodiments, the elongate member defines an inflation lumen.

Alternatively, or additionally, in the above embodiment, the dilation device may further comprise a balloon member connected to the tip member, and wherein the inflation lumen opens into the balloon member.

Alternatively, or additionally, in any of the above embodiments, the elongate member comprises a proximal section having a first diameter and a distal section comprising a second diameter, wherein the second diameter is larger than the first diameter.

Alternatively, or additionally, in any of the above embodiments, the elongate member further comprises a swallow member connected proximate the distal end of the elongate member.

Alternatively, or additionally, in any of the above embodiments, the swallow member weighs about 2 to 100 grams, or about 2 to 5 grams, or between about 2 and 25 grams, or between about 25 grams and about 100 grams.

Alternatively, or additionally, in any of the above embodiments, the swallow member has a dynamic viscosity of between about 150 Pascal seconds and about 250 Pascal seconds.

Alternatively, or additionally, in any of the above embodiments, the elongate member may further define a guidewire lumen.

In still another embodiment, a device for locating strictures comprises an elongate member having a proximal end and a distal end and a stopper attached to the distal end of the elongate member. In at least some of these embodiments, the stricture locating device may further comprise distance markings disposed on the elongate member.

Alternatively, or additionally, in the above embodiment, the stopper comprises a balloon member.

Alternatively, or additionally, in any of the above embodiments, the stopper is cone shaped, and wherein the base of the cone shape is attached to the distal end of the elongate member.

Alternatively, or additionally, in any of the above embodiments, the stopper is shaped as a frustum of a cone, and wherein the base of the frustum of the cone is attached to the distal end of the elongate member.

Alternatively, or additionally, in any of the above embodiments, the stopper is umbrella shaped.

Alternatively, or additionally, in any of the above embodiments, the stopper comprises one or more arms connected together to form a ball-like shape.

Alternatively, or additionally, in any of the above embodiments, the stopper includes a living hinge.

Alternatively, or additionally, in any of the above embodiments, the elongate member further includes a frangible portion.

Alternatively, or additionally, in any of the above embodiments, the stopper contains one or more frangible portions.

Alternatively, or additionally, in any of the above embodiments, the stricture locating device may further comprise a marker adjustably disposed on the elongate member.

In another embodiment, a method of locating a stricture comprises inserting a stricture locating device into an esophagus of a patient. In at least some of these embodiments the stricture locating device may comprise an elongate member having a proximal end and a distal end, a stopper attached to the distal end of the elongate member, and distance markings disposed on the elongate member. Additionally, the method may include pushing the stopper past the stricture and retracting the stricture locating device until the stopper contacts the stricture.

Alternatively, or additionally, in the above embodiment, the stricture locating device further comprises a marker adjustably disposed on the elongate hinge, the method further comprised adjusting the marker to contact the lips or teeth of the patient.

Alternatively, or additionally, in any of the above embodiments, the method may further include sliding a dilation device over the elongate member, where the dilation device comprises a shaft having a proximal end and a distal end, the shaft defining an inflation lumen and a guidewire lumen, and a balloon member disposed proximate the distal end of the shaft, wherein an interior of the balloon member is in fluid communication with the inflation lumen of the shaft, and wherein sliding a dilation device over the elongate member comprises inserting the elongate member into the guidewire lumen of the shaft.

Alternatively, or additionally, in any of the above embodiments, the method may further comprise disposing the balloon member of the dilation device proximate the stricture, and inflating the balloon member by delivering inflation media through the inflation lumen of the shaft.

In still another embodiment, a dilation device for insertion into an esophagus comprises an elongate member having a proximal end and a distal end, wherein the elongate member has a tip member bent at an angle relative to the elongate member disposed at the distal end of the elongate member, a reservoir connected to the proximal end of the elongate member, and a valve disposed between the proximal end of the elongate member and the reservoir. Additionally, in at least some embodiments, the elongate member defines an inflation lumen.

Alternatively, or additionally, in the above embodiment, the dilating device may further comprise a balloon member connected to the tip member, and wherein the inflation lumen opens into the balloon member.

Alternatively, or additionally, in any of the above embodiments, the elongate member comprises a proximal section having a first diameter and a distal section having a second diameter, wherein the second diameter is greater than the first diameter.

Alternatively, or additionally, in any of the above embodiments, the elongate member comprises a non-compliant material.

Alternatively, or additionally, in any of the above embodiments, at least a portion of the elongate member has an inner diameter of between about 0.7 inches and about 1.2 inches.

Alternatively, or additionally, in any of the above embodiments, at least a portion of the elongate member has an inner diameter of about 0.9 inches.

Alternatively, or additionally, in any of the above embodiments, a distal portion of the elongate member is tapered.

Alternatively, or additionally, in any of the above embodiments, at least a portion of the tip member is rigid.

In still another embodiment, a method for dilating a stricture of a patient comprises inserting a dilating device into an esophagus. In at least some embodiments, the dilating device comprises an elongate member having a proximal end and a distal end and defining an inflation lumen, and a balloon member disposed on the distal end of the elongate member with an interior of the balloon member in fluid communication with the inflation lumen defined by the elongate member. The method may further include positioning the balloon member in the esophagus closer to a stomach of the patient than a stricture, and inflating the balloon member. Additionally, the method may include retracting the balloon member through the stricture.

Alternatively, or additionally, in the above embodiment, the balloon member may have a distal end and proximal end, and the proximal end of the balloon may be tapered.

Alternatively, or additionally, in any of the above embodiments, the dilating device may further include distance markings disposed proximate the proximal end of the elongate member.

Alternatively, or additionally, in any of the above embodiments, the dilating device may further include a marker adjustably disposed on the elongate member.

In still another embodiment, a method for dilating a stricture of a patient comprises inserting a dilating device into an esophagus. In at least some embodiments, the dilating device comprises an elongate sheath member having a proximal end and a distal end, where the distal end is sealed. The method may further include positioning the dilating device in the esophagus such that the dilating device is disposed along a majority of the length of the esophagus. The method may further include inflating the dilating device to dilate the majority of the esophagus.

Alternatively, or additionally, in any of the above embodiments, the dilating device is non-compliant and has a fixed diameter.

Alternatively, or additionally, in any of the above embodiments, the dilating device is compliant.

The above summary is not intended to describe each embodiment or every implementation of the present disclosure. Advantages and attainments, together with a more complete understanding of the disclosure, will become apparent and appreciated by referring to the following description and claims taken in conjunction with the accompanying drawings.

### BRIEF DESCRIPTION OF THE DRAWINGS

The aspects of the disclosure may be further understood in consideration of the following detailed description of various embodiments in connection with the accompanying drawings, in which:

FIG. 1 is a schematic illustration of a patient showing a stricture locating device within the patient's esophagus, in accordance with embodiments of the present disclosure;

FIG. 2 is a depiction of an exemplary stricture locating device, in accordance with embodiments of the present disclosure;

FIG. 3A is an illustration of the stricture locating device of FIG. 2 disposed within an esophagus of a patient;

FIG. 3B is an illustration of the stricture locating device of FIG. 2 disposed within a stricture within an esophagus of a patient;

FIG. 3C is another illustration of the stricture locating device of FIG. 2 disposed within an esophagus of a patient;

FIGS. 4A and 4B are illustrations of a distal portion of a stricture locating device including frangible portions, in accordance with embodiments of the present disclosure;

FIGS. 5A and 5B are illustrations of a distal portion of a stricture locating device including frangible portions, in accordance with embodiments of the present disclosure;

FIGS. 6A and 6B are perspective views of example stoppers of a stricture locating device, in accordance with embodiments of the present disclosure;

FIGS. 7A and 7B are plan views of an example stopper of a stricture locating device including a living hinge, in accordance with embodiments of the present disclosure;

FIGS. 8A and 8B are views of the stricture locating device of FIG. 2 including a guide catheter;

FIG. 9 is a depiction of an exemplary dilation device, in accordance with embodiments of the present disclosure;

FIG. 10 is a cross-sectional view of the dilation device of FIG. 9, in accordance with embodiments of the present disclosure;

FIGS. 11A-11D are depictions of an example dilating procedure for dilating a stricture using the stricture locating device of FIG. 2 and the dilation device of FIG. 9, in accordance with embodiments of the present disclosure;

FIGS. 12A-12C are depictions of an example dilating procedure for dilating a stricture using an exemplary dilating device, in accordance with embodiments of the present disclosure;

FIG. 13 is a plan view of an example distal portion of a dilating device, in accordance with embodiments of the present disclosure;

FIG. 14 is a depiction of an exemplary dilating device, in accordance with embodiments of the present disclosure;

FIG. 15 is a plan view of an exemplary distal portion of the dilating device of FIG. 14 including a rigid tip portion, in accordance with embodiments of the present disclosure;

FIG. 16 is a plan view of an exemplary distal portion of the dilating device of FIG. 14 including a curved rigid tip portion, in accordance with embodiments of the present disclosure;

FIG. 17 is a plan view of an exemplary distal portion of the dilating device of FIG. 9 including a rigid tip portion, in accordance with embodiments of the present disclosure;

FIG. 18 is a plan view of an exemplary distal portion of an exemplary dilating device including a rigid tip portion, in accordance with embodiments of the present disclosure; and

FIG. 19 is a plan view of an exemplary distal portion of the dilating device of FIG. 9 including a swallow member, in accordance with embodiments of the present disclosure.

While the aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

Definitions of certain terms are provided below and shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a,” “an,” and “the” include or otherwise refer to singular as well as plural referents, unless the content clearly dictates otherwise. As used in this specification and the appended claims, the term “or” is generally employed to include “and/or,” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

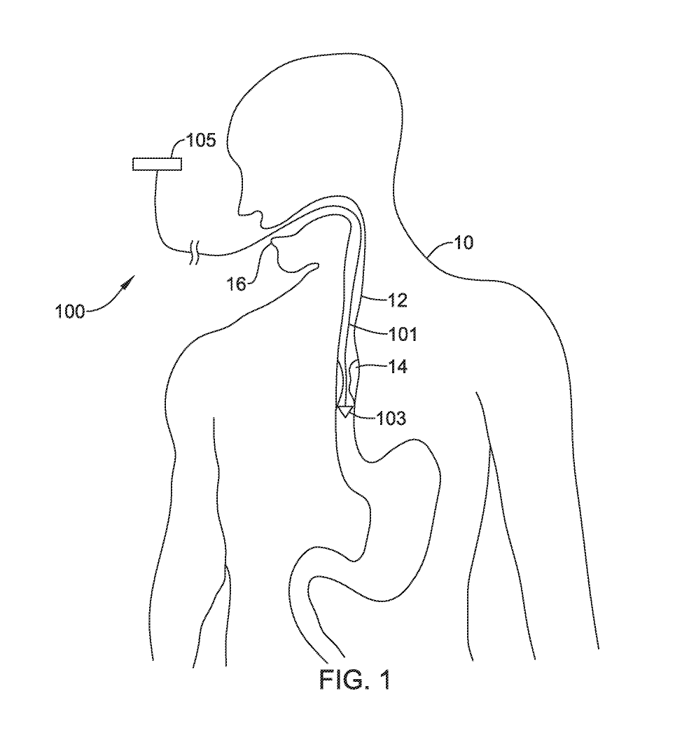

FIG. 1 is a schematic illustration of a torso of a patient 10. Patient 10 includes an esophagus 12 with stricture 14 and mouth 16. FIG. 1 also depicts stricture locating device 100 at least partially disposed within esophagus 12. As shown, stricture locating device 100 may include elongate member 101, stopper 103, and handle 105. Patient 10 may swallow or otherwise extend stopper 103 and a portion of elongate member 103 down esophagus 12. Patient 10 may continue extending stricture locating device 100 down esophagus 12 until stopper 103 passes stricture 14. Patient 10 may determine that stopper 103 has passed stricture 14, for example, by encountering increased resistance to continued extension of stricture locating device 100 down esophagus 12 or after extending a predetermined length of stricture locating device 100 down esophagus 12.

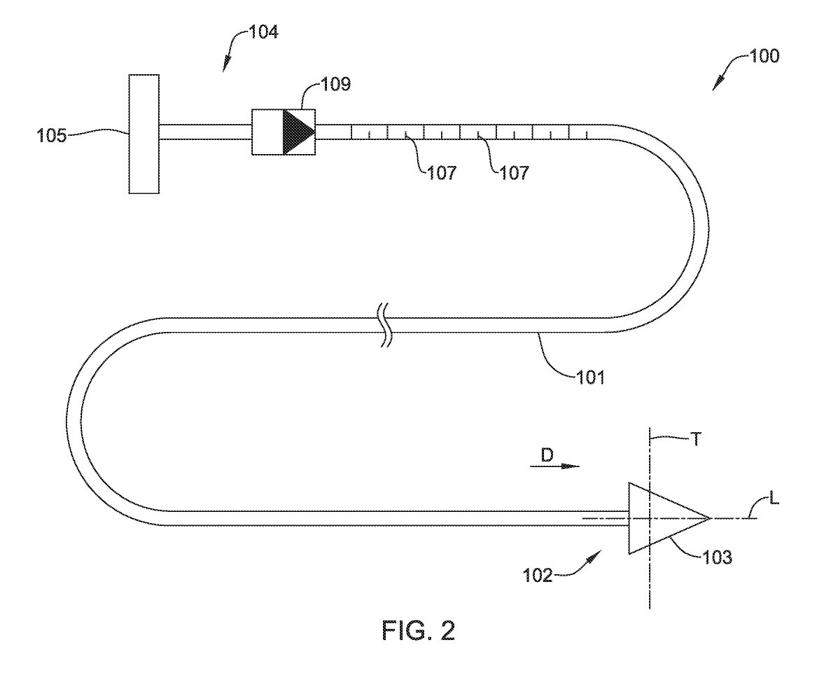

FIG. 2 illustrates an example embodiment of stricture locating device 100. As seen in FIG. 1, stricture locating device 100 may include elongate member 101, stopper 103, and handle 105. Additionally in some embodiments, stricture locating device 100 may further include distance markings 107 and marker 109. In some cases, elongate member 101 may be a solid body, for instance similar to one or more guidewires known in the art. However, in other cases, elongate member 101 may be hollow and define a lumen extending through at least a portion of elongate member 101. Generally, elongate member 101 may be made from any suitable biocompatible or biostable materials. Examples of the biostable metal materials may include, but are not limited to, stainless steel, tantalum, tungsten, niobium, platinum, nickel-chromium alloys, cobalt-chromium alloys such as Elgiloy® and Phynox®, nitinol (e.g., 55% nickel, 45% titanium), and other alloys based on titanium, including nickel titanium alloys, or other suitable metals, or combinations or alloys thereof. Some suitable biostable polymeric materials include, but are not necessarily limited to, polyamide, polyether block amide, polyethylene, polyethylene terephthalate, polypropylene, polyvinylchloride, polyurethane, polytetrafluoroethylene, polysulfone, and copolymers, blends, mixtures or combinations thereof. Examples of suitable bioabsorbable materials may include polymers, such as poly-L-lactide (PLLA), polyglycolide (PGA), polylactide (PLA), poly-D-lactide (PDLA), polycaprolactone, polydioxanone, polygluconate, polylactic acid-polyethylene oxide copolymers, modified cellulose, collagen, poly(hydroxybutyrate), polyanhydride, polyphosphoester, poly(amino acids), and combinations thereof.

Stopper 103 may also be comprised of one or more biostable or biocompatible polymeric materials, such as any of those described with respect to elongate member 101. In other embodiments, however, stopper 103 may be made from one or more food products which may be broken down and metabolized by the digestive system of patient 10. Stopper 103 may be attached to elongate member 101 at distal end 102 of elongate member 101, for example by laser bonding, heat bonding, or by one or more adhesives.

In some embodiments, stopper 103 may be compressible. For instance, stopper 103 may have a transverse extent and a longitudinal extent, where the transverse direction is defined as parallel to transverse axis T and the longitudinal direction is defined as parallel to the longitudinal axis L. When net forces act on stopper 103 in directions parallel to the transverse or longitudinal directions, the transverse extent and/or the longitudinal extent of stopper 103 may change. More specifically, in such situations the transverse extent and/or longitudinal extent of stopper 103 may be less than the transverse extent and/or longitudinal extent when no net forces are acting on stopper 103. In some cases, the relative compressibility of stopper 103 may be different in the transverse and longitudinal directions. For instance, in at least some embodiments, stopper 103 may be relatively more compressible under forces acting in the transverse direction than the longitudinal direction. In some particular examples, stopper 103 may be relatively more compressible under forces acting in the transverse direction than under forces acting parallel to the longitudinal axis and acting in the distal direction, as defined by arrow D.

As depicted, in some embodiments, stricture locating device 100 may additionally include one or more distance markings 107. Distance markings 107 may indicate the length of elongate member 101 extending from each marking to the distal end of either elongate member 101 or stopper 103. Accordingly, once patient 10 locates stricture 14, patient 10 may use distance markings 107 to determine how far down esophagus 12 stricture 14 is located. In at least some embodiments, patient 10 may additionally use marker 109 to determine how far down esophagus 12 stricture 14 is located. For instance, marker 109 may be slidably disposed around elongate member 101 so that marker 109 may be adjusted in relation to distance markings 107. In at least some embodiments, marker 109 may further include a securing mechanism such that once marker 109 has been adjusted to a desired location, marker 109 may be secured to elongate member 101 to maintain the position of marker 109 in relation to elongate member 101. Once patient 10 has located stricture 14, patient 10 may slide marker 109 to contact mouth 16, the teeth of patient 10, or some other reference point on the body of patient 10. During and after extraction of stricture locating device 100 from esophagus 12, marker 109 may retain its position, thereby allowing patient 10 to determine how far down esophagus 12 stricture 14 is located relative to mouth 16, the teeth of patient 10, or some other bodily reference point. By knowing the specific location of stricture 14 within esophagus 12, patient 10 may better treat stricture 14.

Handle 105 may be disposed at proximal end 104 of stricture locating device 100. Handle 105 may allow for easier grasping of stricture locating device 100 by patient 10. Additionally, handle 105 may prevent marker 109 from sliding proximally off of elongate member 101. In embodiments other than that shown in FIG. 1, handle 105 may take various other forms. In some additional embodiments, such as where elongate member 101 acts as a guidewire to another device, stricture locating device 100 may not include handle 105.

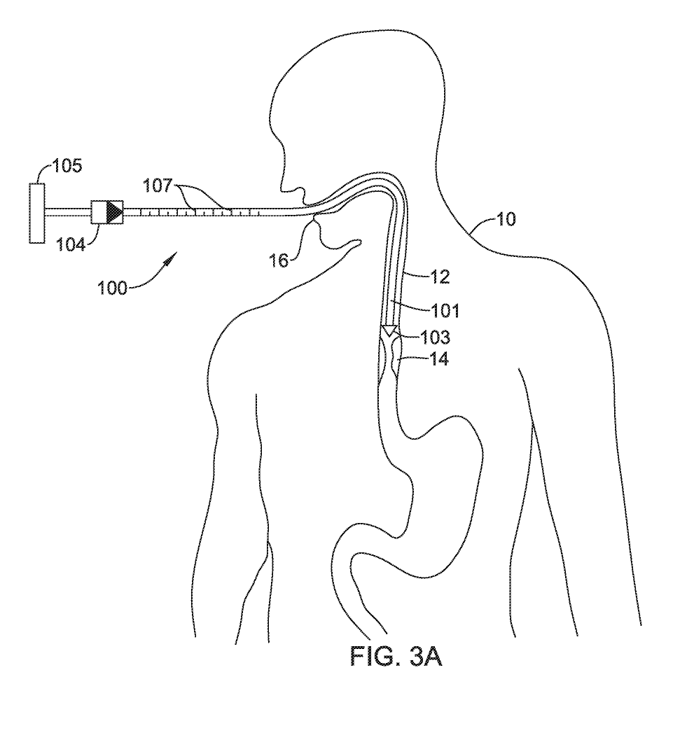

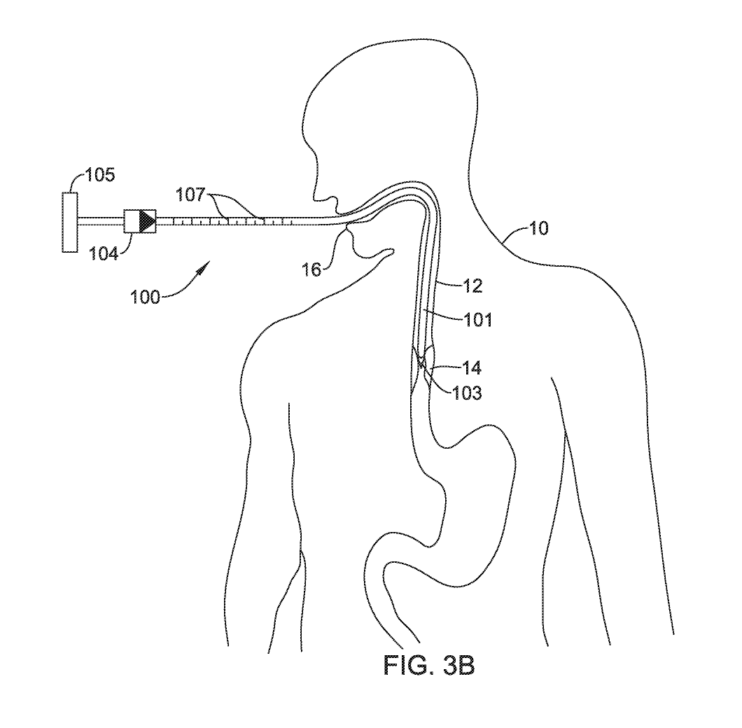

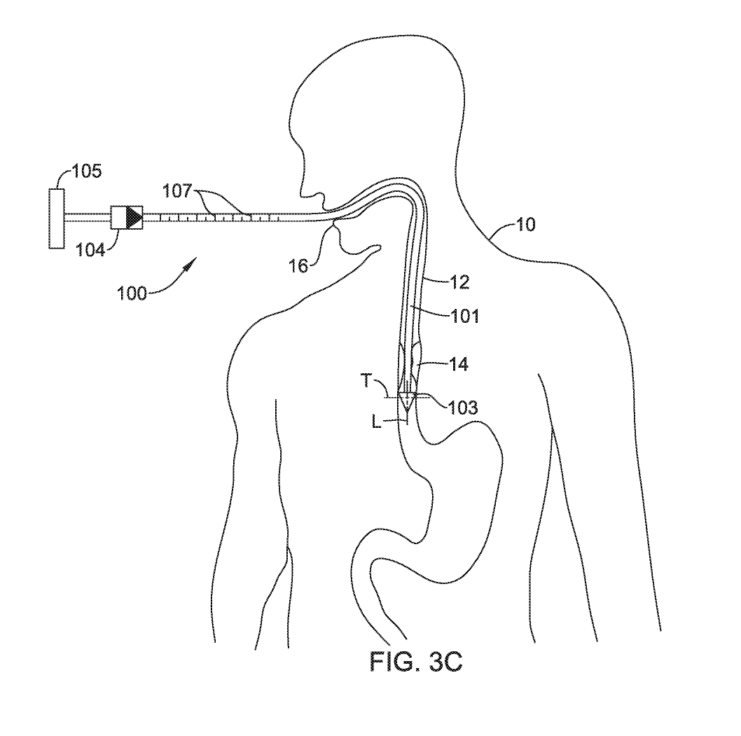

FIGS. 3A-3C generally depict how patient 10 may use stricture locating device 100. As shown in FIG. 3A, patient 10 may begin by swallowing or otherwise inserting stricture locating device 100 into esophagus 12. FIG. 3B depicts stopper 103 disposed at least partially within stricture 14. As patient 10 continues to push stricture locating device 100 into esophagus 12, stopper 103 encounters stricture 14. As stopper 103 enters stricture 14, the sides of stopper 103 may compress so that the transverse extent of stopper 103 is smaller than the transverse extent of stopper 103 when no net forces are acting on stopper 103, as seen in FIG. 3B. This compressibility may allow for stopper 103 to pass through stricture 14, at least in one direction.

FIG. 3C depicts stopper 103 located beyond stricture 14. As patient 10 continues to push stricture locating device 100 into esophagus 12, stopper 103 may continue to extend down esophagus 12 beyond stricture 14. When patient 10 extends a predetermined length of stricture locating device 100 down esophagus 12, patient 10 may determine that stopper 103 is disposed beyond stricture 14. In some instances, patient 10 may encounter resistance to pushing stricture locating device 100 down esophagus 12, which may relent after continuing to push stricture locating device 100 down esophagus 12. After determining the resistance relented, patient 10 may determine that stopper 103 is disposed beyond stricture 14.

Once patient 10 determines that stopper 103 is disposed beyond stricture 14 in esophagus 12, patient 10 may retract stricture locating device 100, including stopper 103, until encountering resistance. Once patient 10 encounters resistance to retracting stricture locating device 100, stopper 103 may be disposed at the end of stricture 14, as depicted in FIG. 3C. Patient 10 may then adjust marker 109 until marker 109 touches mouth 16, and may secure the position of marker 109 relative to elongate member 101.

After adjusting marker 109, patient 10 may then continue to retract stricture locating device 100 all the way out of esophagus 12. For instance, by applying increased retraction force, patient 10 may cause stopper 103 to compress or deform in order to pull stopper 103 back through stricture 14.

In other embodiments, instead of retracting stopper 103, patient 10 may only retract elongate member 101. For instance, in some embodiments where stopper 103 is attached to elongate member 101 with one or more adhesives, the one or more adhesives may be soluble adhesives. More specifically, the one or more soluble adhesives may be soluble in the aqueous environment of esophagus 12. Accordingly, once stopper 103 is exposed to the aqueous environment of esophagus 12, the adhesives holding stopper 103 to elongate member 101 may begin to break down. With enough time in esophagus 12 or enough retraction force, the adhesive bond between stopper 103 and elongate member 101 may break. In embodiments where elongate member 101 defines a lumen, and where the lumen extends all the way to where stopper 103 is bonded to elongate member 101, an appropriate solvent may be delivered to the lumen. As the solvent contacts the bond between stopper 103 and elongate member 101, the bond may weaken, thereby allowing stopper 103 to be detached from elongate member 101.

After detachment from elongate member 101, stopper 103 may fall into the stomach of patient 10 and pass through the digestive system of patient 10, ultimately being excreted from patient 10. In embodiments where stopper 103 is made from one or more food products, patient 10 may metabolize stopper 103, which may beneficially provide nutrients to patient 10.

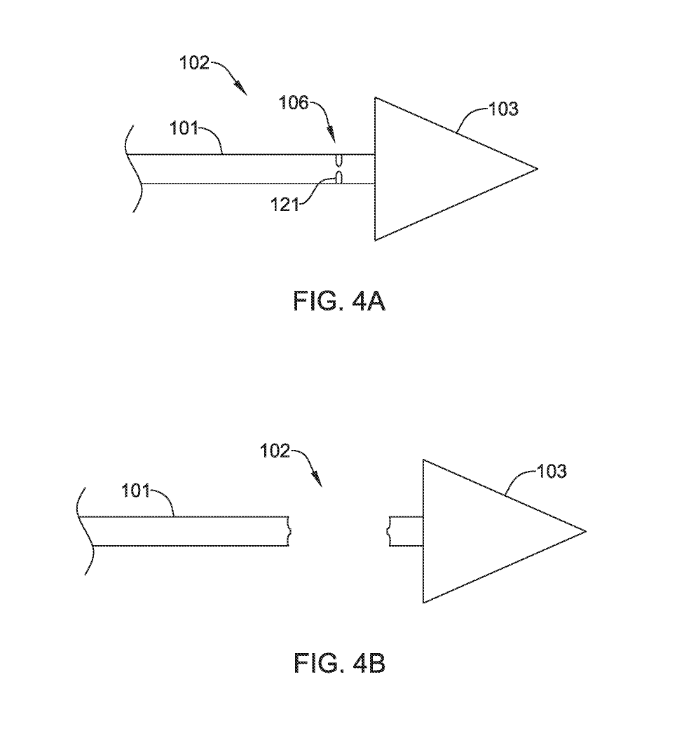

In additional or alternative embodiments, elongate member 101 may include one or more frangible portions, as depicted in FIGS. 4A and 4B. FIG. 4A depicts an embodiment of stricture locating device 100 where elongate member 101 includes one or more frangible features 121 in region 106 disposed near proximal end 102 of stricture locating device 100. In some embodiments, frangible features 121 may be perforations through elongate member 101. In additional or alternative embodiments, frangible features 121 may comprise recesses in the material of elongate member 101. In still more additional or alternative embodiments, frangible features 121 may comprise locations where elongate member 101 has been crimped. Generally, such frangible features 121 may mechanically weaken elongate member 101 in the vicinity of region 106. When frangible features 121 are included, a sufficient retraction force applied to elongate member 101 while stopper 103 is disposed beyond stricture 14 in esophagus 12 may cause elongate member 101 to break in the vicinity of region 106, thereby separating elongate member 101 from stopper 103. FIG. 4B depicts elongate member 101 and stopper 103 after elongate member 101 has been separated from stopper 103 by the breaking of elongate member 101 in the vicinity of region 106. In these embodiments, stopper 103 may then fall into the stomach of patient 10 and ultimately be excreted or metabolized.

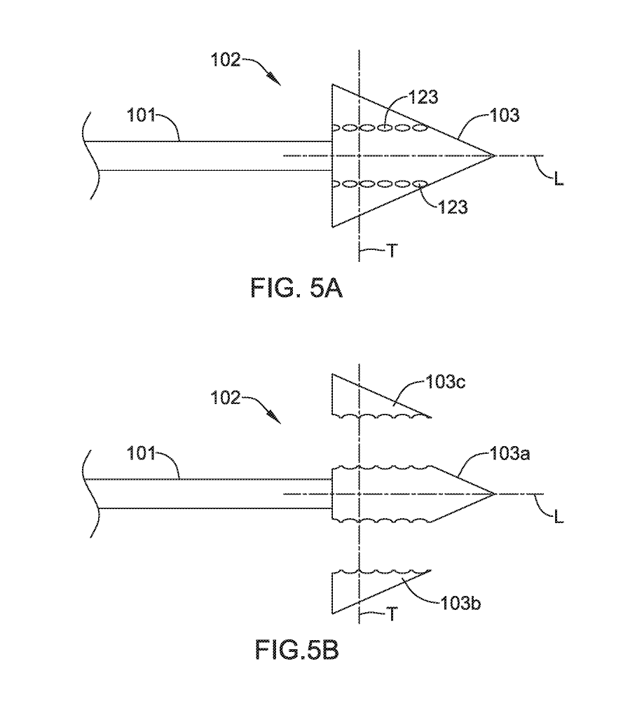

In some examples, instead of, or in addition to, elongate member 101 having one or more frangible features, stopper 103 may have one or more frangible features, as depicted in FIGS. 5A and 5B. FIG. 5A depicts distal end 102 of an embodiment of stricture locating device 100 where elongate stopper 103 includes one or more frangible features 123. In some of these embodiments, frangible features 123 may be perforations through stopper 103. In additional or alternative embodiments, frangible features 123 may comprise recesses in the material of stopper 103. In still more additional or alternative embodiments, frangible features 123 may comprise locations where stopper 103 has been crimped. Generally, such frangible features 123 may mechanically weaken stopper 103 in the vicinity of the frangible features 123. When frangible features 123 are included, a sufficient retraction force applied to elongate member 101 while stopper 103 is disposed beyond stricture 14 in esophagus 12 may cause stopper 103 to break in the vicinity of the frangible features 123. As can be seen in FIG. 5B, in embodiments where stopper 103 includes multiple frangible features 123, stopper 103 may break into multiple segments 103a-103c. In some embodiments, at least a portion of stopper 103 may remain attached to elongate member 101, such as segment 103a. However, after one or more segments 103b, 103c break off from segment 103a, segment 103a may have a transverse extent that is less than the transverse extent of stopper 103. Accordingly, segment 103a may then be able to be retracted through stricture 14 and removed from esophagus 12 along with the rest of stricture locating device 100. Segments 103b, 103c, on the other hand, may fall into the stomach of patient 10 and subsequently be excreted or metabolized.

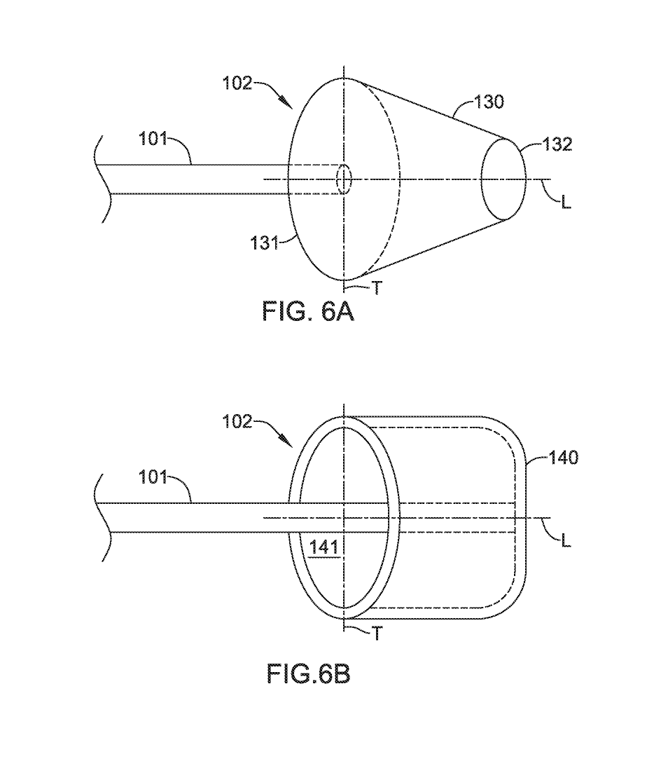

FIGS. 1A-5B have depicted stopper 103 in a plan view and as generally having a triangular shape when viewed in plan. Accordingly, stopper 103 may have a generally cone or pyramid shape when viewed in three-dimensions. However, this disclosure contemplates that stopper 103 may take other shapes. For instance, FIGS. 6A and 6B depict alternative embodiments of a stopper that may be connected to elongate member 101. FIG. 6A depicts stopper 130 in a perspective view. As seen, stopper 130 may have a shape similar to a frustum of a cone, with the base 131 of the frustum attached to elongate member 101, and the top 132 of the frustum extending distally away from elongate member 101. As stopper 130 extends distally away from elongate member 101, stopper 130 may taper down to top 132. FIG. 6B depicts stopper 140 also in a perspective view. As seen, stopper 140 may generally have an umbrella or cup shape, where stopper 140 defines cavity 141. As with stopper 103, stoppers 130 and 140 may also be relatively more compressible or deformable in the transverse direction than in the longitudinal direction.

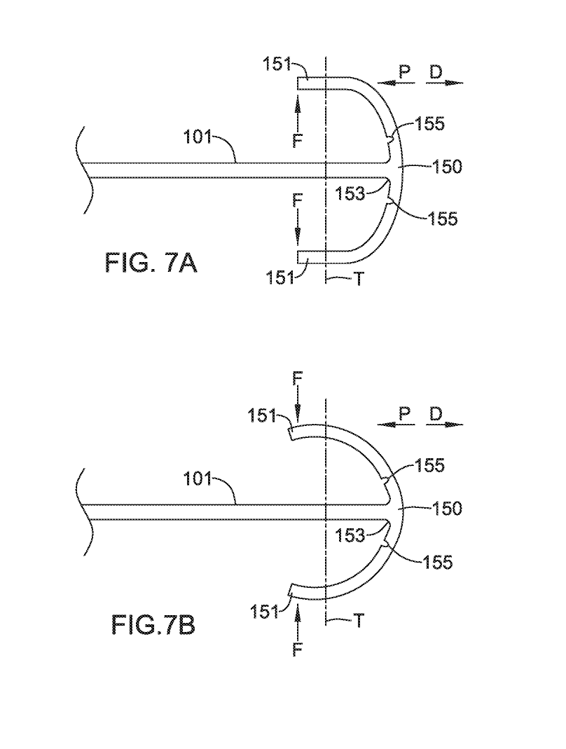

FIGS. 7A and 7B depict yet another embodiment of a stopper, stopper 150. Stopper 150 may include arms 151 that curl back away from connection point 153 toward the proximal end of elongate member 101, as defined by arrow P. Arms 151 may additionally include living hinges 155. Living hinges 155 may allow arms 151 to bend in a first direction but not in the opposite of the first direction, or at least to a lesser extent in the opposite of the first direction. For instance, living hinges 155 may resist forces acting parallel to the transverse axis T and outward away from elongate member 101, as depicted by arrows F in FIG. 7A. However, living hinges 155 may allow for bending of arms 151 under forces acting parallel to the transverse axis T and inward toward elongate member 101, as depicted by arrows F in FIG. 7B. Accordingly, as seen in FIG. 7A, arms 151 have a first transverse extent and, as seen in FIG. 7B, arms 151 have a second transverse extent that is less than the first transverse extent. In these embodiments, as stopper 150 is passed through a stricture, such as stricture 14, the stricture may apply forces in a similar direction to arrows F in FIG. 7B, thereby compressing stopper 150 into a smaller transverse extent. Once past the stricture, stopper 150 may assume its unstressed state and may have a greater transverse extent than when passing through the stricture. As stopper is retraced, stricture may apply forces in the longitudinal direction (not shown in FIGS. 7A and 7B) and/or in the direction of arrows F in FIG. 7A, which living hinges 155 may resist. Accordingly, living hinges 155 may prevent stopper 150 from being retracted back through the stricture.

In still other embodiments, stopper 103 may take on any desirable shape that may allow passage of stopper 103 past a stricture in a first direction, yet generally prevent or resist retraction of stopper 103 back through the stricture in the opposite of the first direction. In at least some embodiments, stopper 103 may not have different compressibility in different directions. For instance, stopper 103 may be a round, ball shaped stopper that is uniformly compressible. In such embodiments, patient 10 may experience resistance as patient 10 advances stricture locating device 100, including the round, ball shaped stopper, through stricture 14. Once the resistance has relented, patient 10 may determine that the round, ball shaped stopper has pass beyond stricture 14. Accordingly, as patient 10 retracts stricture locating device 100, patient 10 may only retract stricture locating device 100 until patient 10 encounters resistance, indicating that the round, ball shaped stopper has encountered the far edge of stricture 14. Patient 10 may then determine how far down esophagus 12 stricture 14 is located. In still other embodiments, stopper 103 may be a balloon member. For instance, patient 10 may insert stricture locating device 100 into esophagus 12 and advance stopper 103 beyond stricture 14. Once patient 10 is confident stopper 103 is disposed beyond stricture 14, patient 10 may inflate stopper 103. When stopper 103 is inflated, patient 10 may retract stricture locating device 100 until encountering resistance.

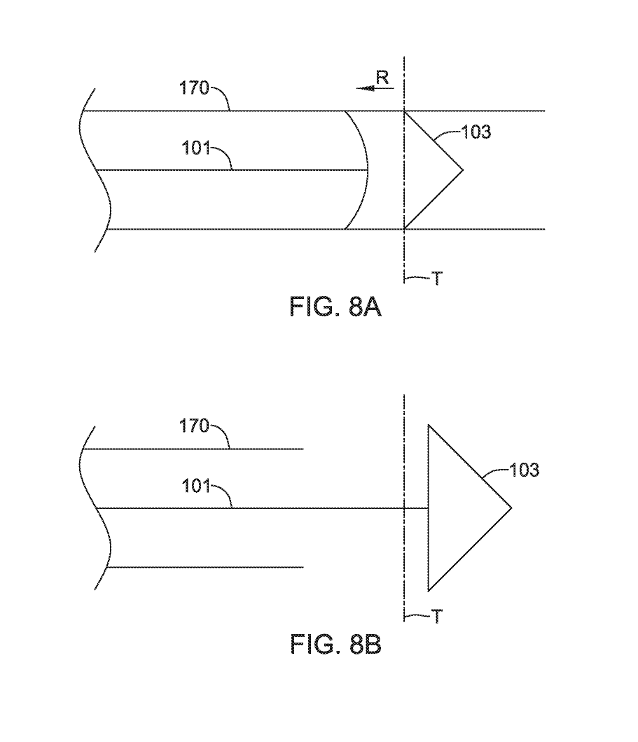

In some additional or alternative embodiments, elongate member 101 and stopper 103 may be delivered to stricture 14 inside of a guide catheter. FIGS. 8A and 8B depict an embodiment of stricture locating device 100 which further includes guide catheter 170. In embodiments where stricture locating device 100 includes guide catheter 170, patient 10 may insert guide catheter 170 containing elongate member 101 and stopper 103 into esophagus 12. When disposed within guide catheter 170, stopper 103 may be in a compressed state and have a smaller transverse extent than when in an uncompressed state. This may allow guide catheter 170, containing elongate member 101 and stopper 103 to more easily pass through stricture 14. Once disposed beyond stricture 14, guide catheter 170 may be retracted, for instance in the direction of arrows R in FIG. 8A, to expose stopper 103. FIG. 8B depicts stopper 103 after guide catheter 170 has been retracted. Once guide catheter 170 has been retracted, stopper 103 may revert to an uncompressed state where stopper 103 has a larger transverse extent, as seen in FIG. 8B. For example, stopper 103 may have a greater transverse extent than when compressed within guide catheter 170. Once guide catheter 170 has been retraced to expose stopper 103, patient 10 may attempt to retract stricture locating device 100, including stopper 103. As stopper 103 encounters stricture 14, patient 10 may encounter resistance to retracting stricture locating device 100, thereby indicating that patient 10 has located stricture 14.

In some additional embodiments, after determining that stopper 103 is disposed at stricture 14, patient 10 may employ a second stricture locating device to locate the top of stricture 14. For instance, patient 10 may advance a second stricture locating device into esophagus 12. Patient 10 may advance the second stricture locating device, including a second stopper, until patient 10 encounters resistance to further advancement of the second stricture locating device. Patient 10 may then adjust a second marker, located on the second stricture locating device, to mouth 16 and secure the second marker. In this manner, patient 10 may determine the positions of both the bottom of stricture 14 and the top of stricture 14, and may further determine a length of stricture 14 by taking the difference of the determined positions.

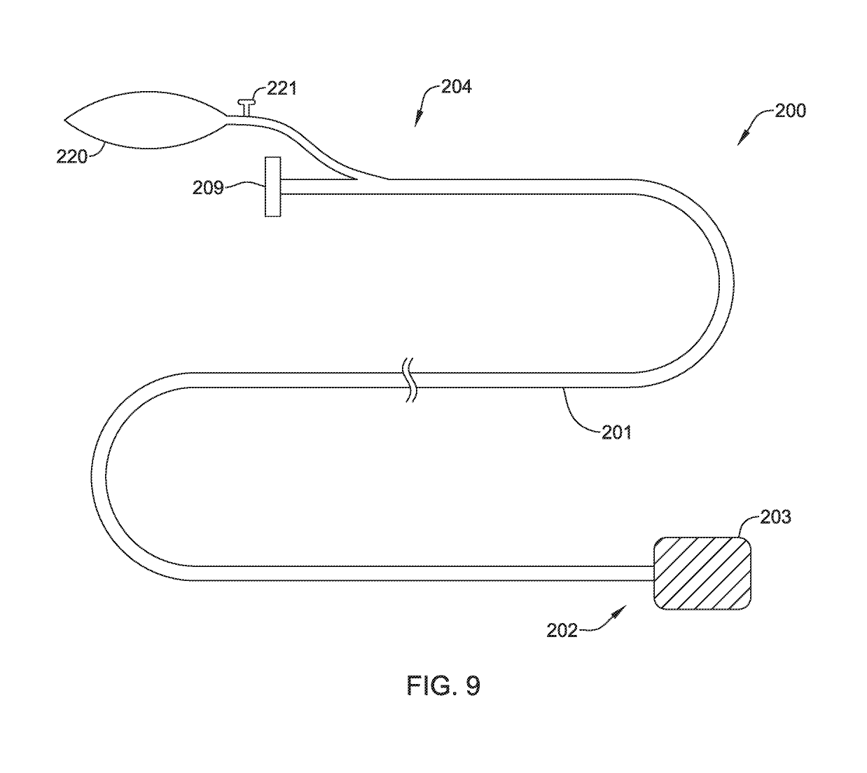

Instead of retracting stricture locating device 100 once patient 10 has located stricture 14, patient 10 may insert another medical device into esophagus 12 for treatment of stricture 14. FIG. 9 depicts an example dilation device 200 that may be used in conjunction with stricture locating device 100 for treating stricture 14. As seen in FIG. 9, dilation device 200 may comprise elongate member 201 and balloon member 203 connected to elongate member 201 near distal end 202. Handle 209 may be connected to elongate member 201 near proximal end 204. Elongate member 201 may be a generally flexible, non-compliant body, for instance similar to a catheter, and may have one or more lumens extending at least partway along the length of elongate member 201. Elongate member 201 may be relatively thin to fit within esophagus 12. For instance, elongate member 201 may have a diameter of between about 0.05 inches (1.27 mm) to about 0.15 inches (3.81 mm). In some embodiments, elongate member 201 includes at least a guidewire lumen. The guidewire lumen may extend all the way through elongate member 201, including through balloon member 203.

In these embodiments, balloon member 203 may be an annular balloon disposed around distal end 202 of dilation device 200. In some embodiments, balloon member 203 may be comprised of one or more materials such as silicone, thermoplastic polyurethane (TPU), SIBS (poly styrene-isobutylene-styrene block copolymer), polyurethane, SEBS styrene ethylene butylene styrene block copolymer, other styrenic block copolymers, or other suitable materials. In at least some embodiments, balloon member 203 may be compliant, such that balloon member 203 stretches as more inflation media is delivered into balloon member 203. However, in other embodiments, balloon member 203 may be non-compliant and may have a static, defined volume and/or diameter.

Elongate member 201 may additionally define an inflation lumen that is in communication with an interior of balloon member 203. A user, such as patient 10, may deliver inflation media through the inflation lumen to inflate balloon member 203. Example inflation media include water, saline solution, and other biologically safe liquids. As seen in FIG. 9, dilation device 200 may further include reservoir 220 connected to elongate member 201 through valve 221. More specifically, reservoir 220 may be in communication with the inflation lumen of elongate member 201 through valve 221. Valve 221 may be a one way valve with release mechanism. For instance, a user, such as patient 10, may deliver inflation media from reservoir 220 into the inflation member of elongate member 201 through valve 221, and the inflation media may not flow back into reservoir 220 through valve 221 when the release mechanism of valve 221 is closed. This may allow the user to deliver inflation media to elongate member 201 to inflate balloon member 203. However, once the release mechanism of valve 221 is opened, valve 221 may no longer act as a one-way valve and may allow inflation media within elongate member 201 and reservoir 220 to flow freely. After the release mechanism of valve 221 is opened, balloon member may deflate to back an unstressed state and squeeze inflation media out of balloon member 203 and back into reservoir 220.

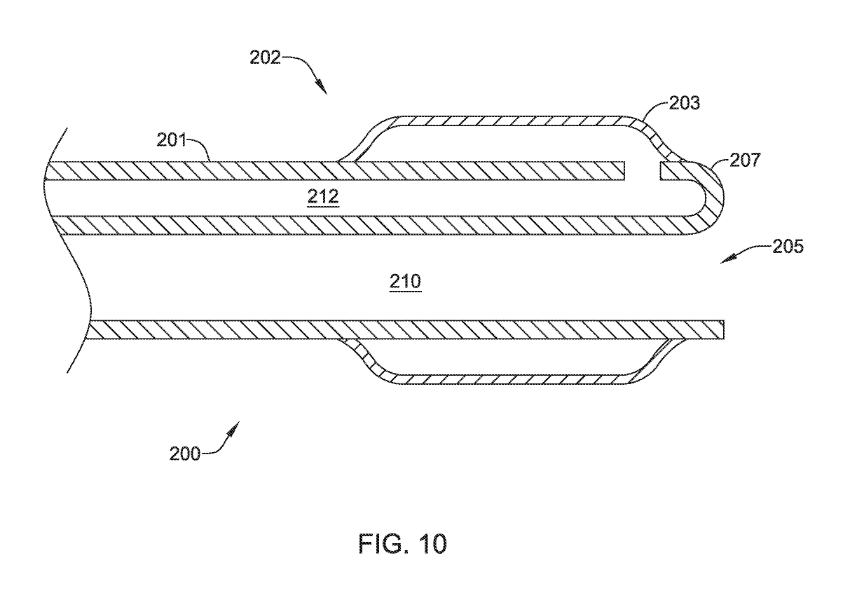

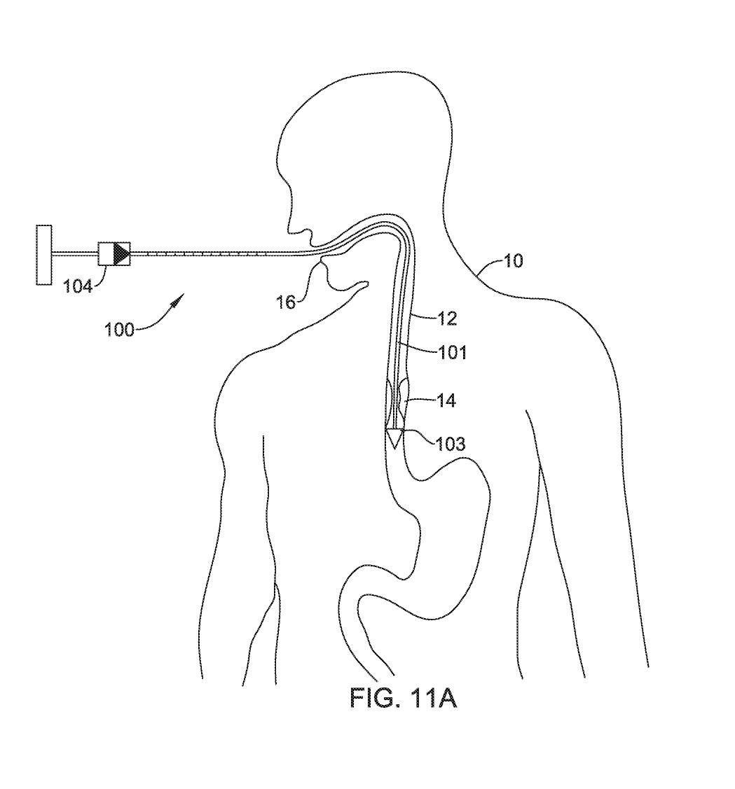

FIG. 10 depicts distal end 202 of dilation device 200 in cross-section with balloon member 203 in an un-inflated state. As seen in FIG. 10, elongate member 201 may include guidewire lumen 210 extending all the way through balloon member 203 and ending in guidewire port 205. FIG. 10 also depicts inflation lumen 212 that is in communication with the interior of balloon member 203 by way of port 207.

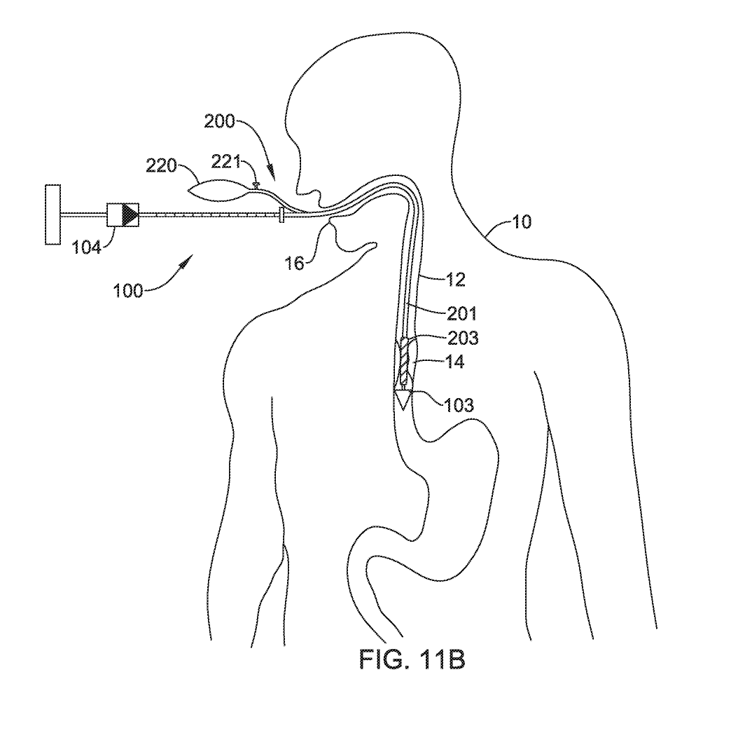

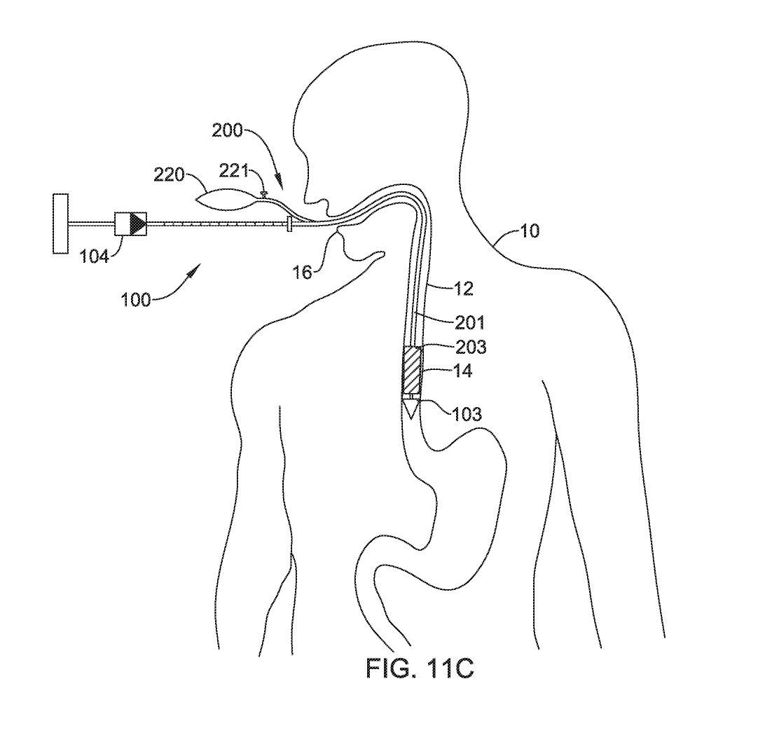

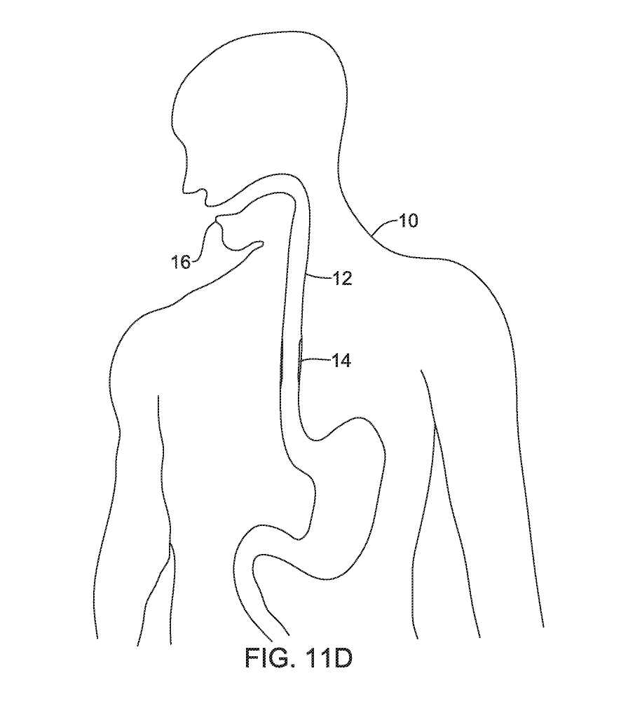

FIGS. 11A-11D depict how dilation device 200 may be used in conjunction with stricture locating device 100 to treat stricture 14. FIG. 11A depicts stopper 103 of stricture locating device 100 disposed just beyond stricture 14, for example where stopper 103 may be located after patient 10 determines the location of stricture 14. Patient 10 may then, as in FIG. 11B, thread dilation device 200 over elongate member 101, for instance by inserting elongate member into guidewire lumen 210. Patient 10 may advance dilation device 200 as far as possible over elongate member 101 until distal end 202 is disposed adjacent stopper 103, as depicted in FIG. 11B. By threading dilation device 200 over elongate member 101 in this manner, patient 10 may be confident that balloon member 203 is disposed within stricture 14. Once dilation device 200 is in position, patient 10 may deliver inflation media from reservoir 220 through inflation lumen 212 and into balloon member 203. Balloon member 203 may then inflate and press on stricture 14 and/or esophagus 12, thereby dilating stricture 14, as in FIG. 11C. After dilation, patient 10 may open the release mechanism of valve 221 to deflate balloon member 203 and may withdraw dilation device 200 and stricture locating device 100 from esophagus 12, leaving stricture 14 dilated as in FIG. 11D.

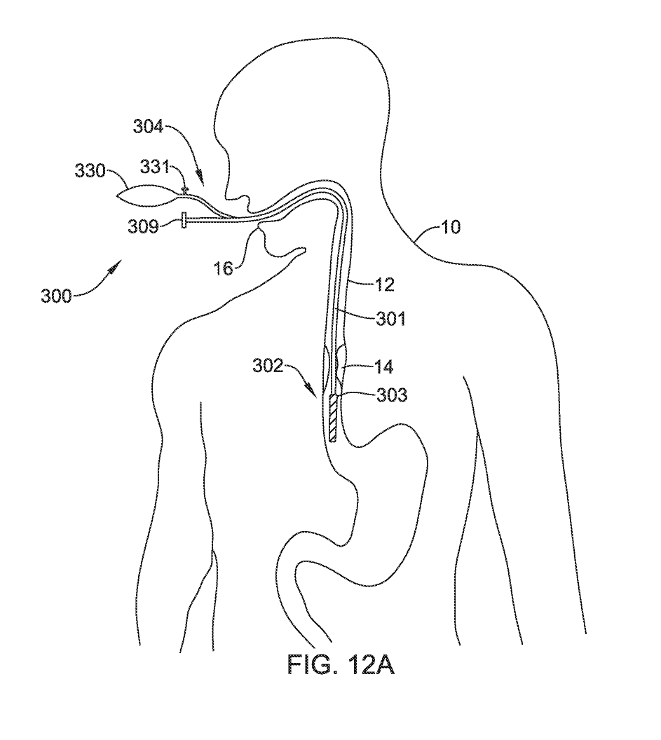

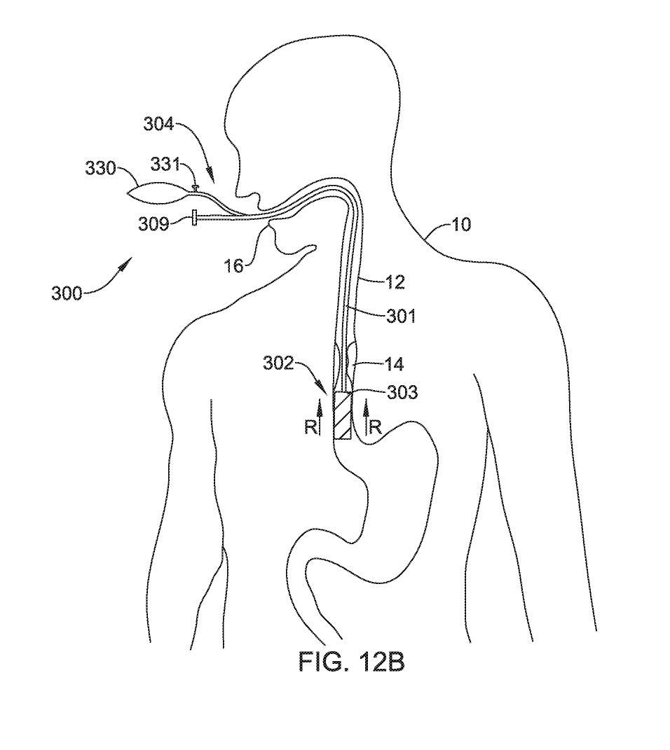

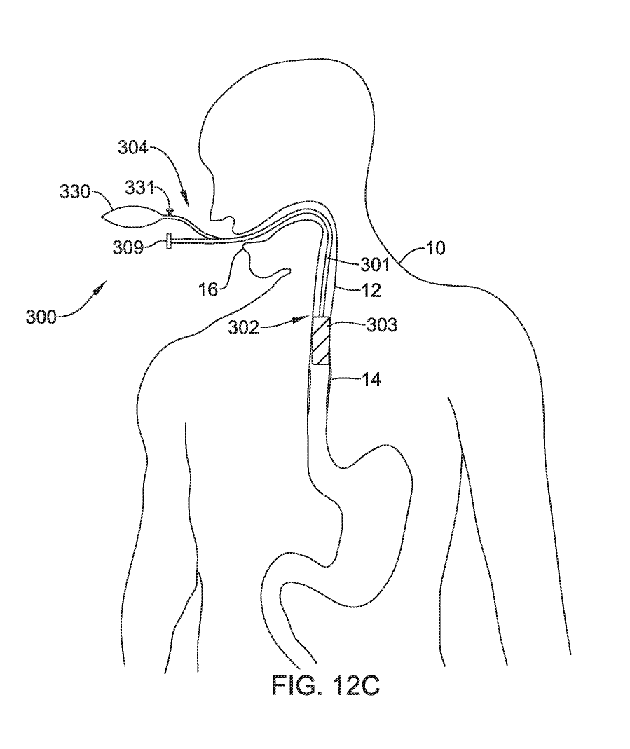

In other embodiments, patient 10 may treat stricture 14 without first locating stricture 14. For instance, patient 10 may utilize a dilation device, such as dilation device 300 depicted in FIGS. 12A-12C. Dilation device 300 may be similar to dilation device 200 depicted in FIG. 9. For example, dilation device 300 may comprise elongate member 301, balloon member 303, handle 309, reservoir 330, and valve 331. Each of these members may be similar to the similarly named members of dilation device 200. However, in at least some embodiments, elongate member 301 may not include a guidewire lumen, as dilation device 300 may not be threaded over a guidewire.

Accordingly, instead of first determining a location of stricture 14, patient 10 may begin by inserting dilation device 300 into esophagus 12, as depicted in FIG. 12A. Patient 10 may insert dilation device 300 into esophagus a predetermined distance—for instance, dilation device 300 may further include one or more distance markings or stops (not shown) disposed near proximal end 304 of dilation device 300. The distance markings or stops may be located a predefined distance from distal end 302 and balloon member 303. Because stricture 14 may be located anywhere within esophagus 12, and patient 10 does not know the location of stricture 14, the predefined distance may be set close to the overall length of an average esophagus. Accordingly, when dilation device 300 is fully inserted into esophagus 12, patient 10 may be confident that balloon member 303 has been pushed beyond stricture 14. For instance, the predetermined distance may be between about eight inches (20.3 cm) to about ten inches (25.4 cm) in different embodiments, which may be optimal for an average sized esophagus.

Once dilation device 300 has been fully inserted, patient 10 may deliver inflation media from reservoir 330 to balloon member 303, causing balloon member 303 to inflate, as depicted in FIG. 12B. Patient 10 may then retract dilation device 300, and specifically balloon member 303 through esophagus 12, for instance in the direction of arrows R. Patient 10 may retract balloon member 303 through stricture 14, thereby dilating stricture 14. FIG. 12C depicts dilation device 300 and stricture 14 after patient 10 has retracted balloon member 303 through stricture 14, leaving stricture 14 dilated.

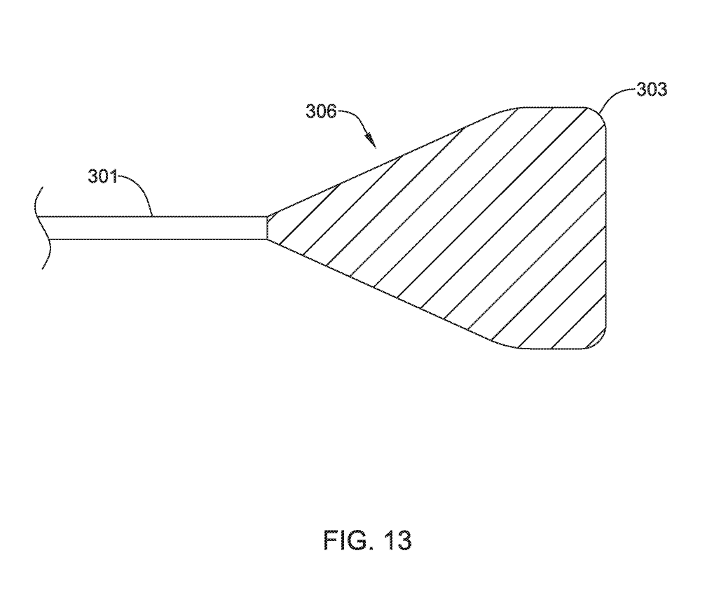

FIG. 13 depicts a close-up of distal end 302 of dilation device 300 including balloon member 303. In examples where patient 10 disposes balloon member 303 beyond stricture 14 and retracts balloon member through stricture 303, balloon member 303 may have one or more features to reduce trauma to esophagus 12 during retraction of balloon member 303 and dilation of stricture 14. For instance, as depicted in FIG. 13, balloon member 303 may be shaped to have a tapered proximal section 306 when inflated. As patient 10 retracts dilation device 300, tapered section 306 may slowly widen stricture 14 as balloon member 303 is retraced through stricture 14.

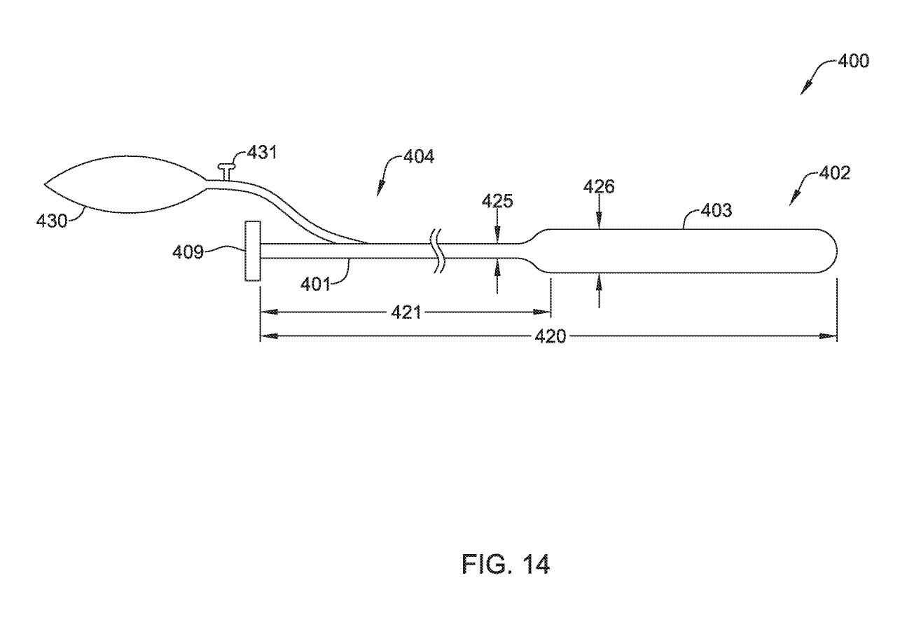

FIG. 14 depicts another embodiment of a dilation device that patient 10 may use to treat stricture 14. FIG. 14 depicts dilation device 400 including proximal elongate member 401 and distal elongate member 403. Dilation device 400 further includes handle 409, reservoir 430, and valve 431. Handle 409, reservoir 430, and valve 431 may all be similar to similarly named members described with respect to dilation device 200. Proximal elongate member 401 and distal elongate member 403 may both be hollow tubular bodies made of a flexible material, such as any of the materials described with respect to elongate member 101 of stricture locating device 100. However, proximal elongate member 401 and distal elongate member 403 may have sufficient rigidity to allow patient to push dilation device 400, but may be flexible enough for patient 10 to easily swallow and to navigate any curves or bends in esophagus 12. Additionally, in at least some embodiments, both of proximal elongate member 401 and proximal elongate member 401 are non-compliant.

Generally, proximal elongate member 401 and distal elongate member 403 may both define a lumen throughout their lengths. In at least some embodiments, proximal elongate member 401 and distal elongate member 403 may be integrally formed such that an outer wall of proximal elongate member 401 expands in diameter to become distal elongate member 403. In other embodiments, however, distal elongate member 403 may be an annular balloon member that is disposed around proximal elongate member 401. In such embodiments, proximal elongate member 401 may extend along the entire length of dilation device 400, and distal elongate member 403 is disposed around proximal elongate member 401. Also in these embodiments, an interior of distal elongate member 403 may be in fluid communication with the lumen of proximal elongate member 401 through one or more ports in proximal elongate member 401.

Additionally, in at least some embodiments, dilation device 400 may have an overall length 420, while distal elongate member 403 may have a length 421, which may be referred to herein as the working length of dilation device 400. Length 421 may generally be similar to that of an average esophagus length so that when distal elongate member 403 is inflated, distal elongate member 403 may dilate the entirety, or at least a substantial portion of, esophagus 12. For instance, length 421 may be between about eight inches (20.3 cm) to about ten inches (25.4 cm) long in different embodiments. Additionally, proximal elongate member 401 and distal elongate member 403 may have different diameters 425 and 426, respectively. In generally, diameter 426 of distal elongate member 403 may be sized such that when distal elongate member 403 is inflated, distal elongate member 403 may dilate esophagus 12. For instance, diameter 426 may be between about 0.7 inches (1.78 cm) and about 1.2 inches (3.05 cm).

Patient 10 may use dilation device 400 in a similar manner to dilation device 300 in that patient 10 may use dilation device 400 without first determining a location of stricture 14. However, instead of requiring patient 14 to retract a balloon member through stricture 14, dilation device 400 may instead dilate the entirety, or at least a substantial portion, of esophagus 12. To start, patient 10 may insert or otherwise extend dilation device 400 into esophagus 12. Dilation device 400 may further include one or more distance markings or stops (not shown) disposed near proximal end 404 of dilation device 400. The distance markings or stops may be located a predefined distance from distal end 402. Because stricture 14 may be located anywhere within esophagus 12, and patient 10 does not know the location of stricture 14, the predefined distance may be set close to the overall length of an average esophagus. Accordingly, when dilation device 400 is fully inserted into esophagus 12, patient 10 may be confident that dilation device 400 extends through the entirety, or at least a substantial portion, of esophagus 12.

Once dilation device has been fully inserted into esophagus 12, patient 10 may deliver inflation media from reservoir 430 into the lumen defined by proximal elongate member 401 and distal elongate member 403. As the inflation media enters the inflation lumen defined by proximal elongate member 401 and distal elongate member 403, distal elongate member 403 may inflate—or expand to its full diameter—and dilate esophagus 12 along the entire length 421. In this manner, patient 10 may dilate stricture 14 without knowing the specific location of stricture 14.

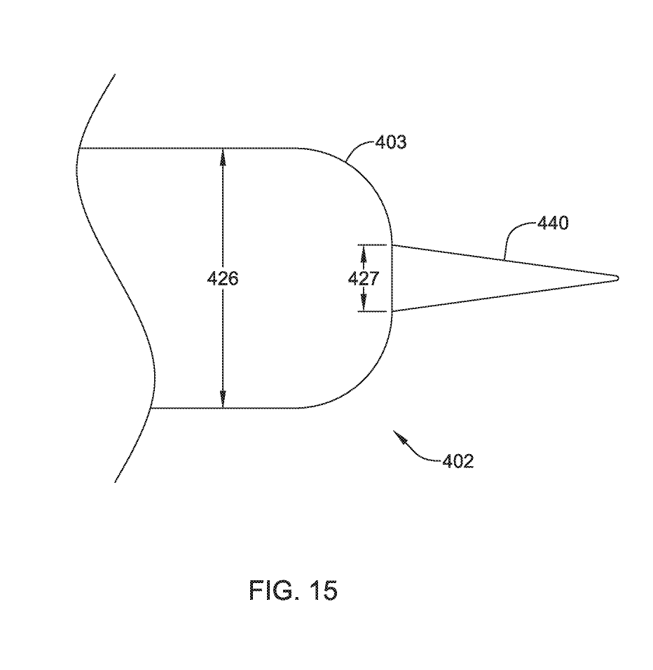

FIG. 15 depicts an alternative distal end 402 of dilation device 400. In some embodiments, distal end 402 of distal elongate member 403 may further include a rigid tip portion 440. In some embodiments, rigid tip portion 440 may be connected onto the distal end of distal elongate member 403 after distal elongate member 403 has been formed, for example through laser welding, heat bonding, or with the use of one or more adhesives. In some embodiments where distal elongate member 403 is an annular balloon member and is disposed over a distal portion of proximal elongate member 401, rigid tip portion 440 may be attached to proximal elongate member 401. Or, in other embodiments, rigid tip portion 440 may be the distal-most portion of proximal elongate member 401. For instance, the distal-most portion of proximal elongate member 401 sealed and modified through one or more processes to impart increased rigidity with respect to the rest of proximal elongate member 401.

As seen in FIG. 15, rigid tip portion 440 may have a relatively small diameter 427 compared to the diameter 426 of distal elongate member 403, although this may not be the case in all embodiments. In other embodiments, rigid tip portion 440 may have a diameter 427 substantially similar to diameter 426. Rigid tip portion 440 may make it easier for patient 10 to swallow or otherwise insert dilation device 400 into esophagus 12. For instance, rigid tip portion may help guide dilation device 400 down esophagus 12 of patient 10 instead of the trachea of patient 10 as patient 10 inserts dilation device 400 into mouth 16. Additionally, in at least some embodiments, rigid tip portion 440 may be tapered as rigid tip portion 440 extends distally away from distal elongate member 403. The taper may additionally make it easier for patient 10 to swallow or otherwise insert dilation device 400 into esophagus 12, or may make it easier for distal elongate member 403 to pass through stricture 12 as patient 10 advances dilation device 400 through esophagus 12.

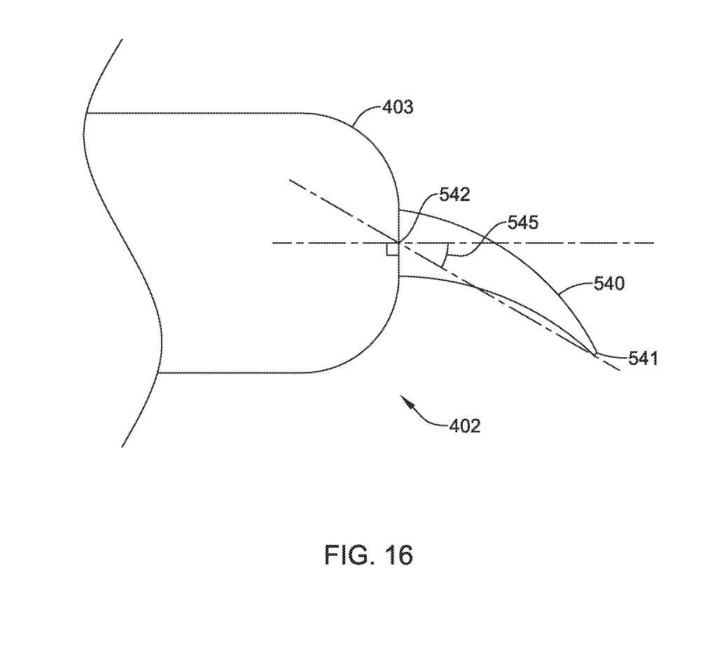

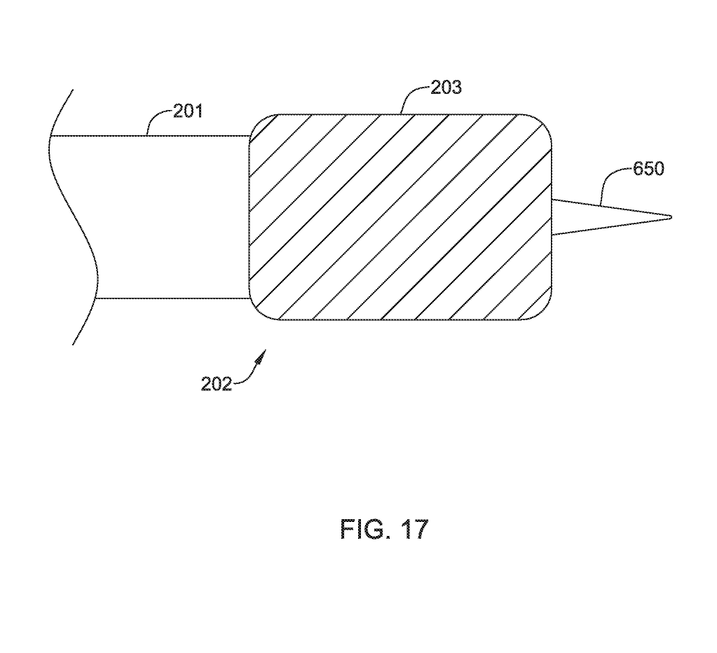

FIG. 16 is another alternative distal end 402 of dilation device 400, and further including rigid tip portion 540. Rigid tip portion 540 may be similar to rigid tip portion 440, except that rigid tip portion 540 may curve as rigid tip portion 540 extends away from distal elongate member 403. For instance tip 541 of rigid tip portion 540 may extend at an angle, as represented by angle 545, away from base 542, where rigid tip portion 540 connects to, or is disposed with respect to, distal elongate member 403. In different embodiments, angle 545 may range from about thirty-five degrees to about seventy degrees. Angling rigid tip portion 540 in this manner may allow for easier swallowing or insertion of dilation device 400 into esophagus 12. For instance, angle 545 may assist patient 10 in inserting dilation device into esophagus 12, as opposed to the trachea of patient 10.

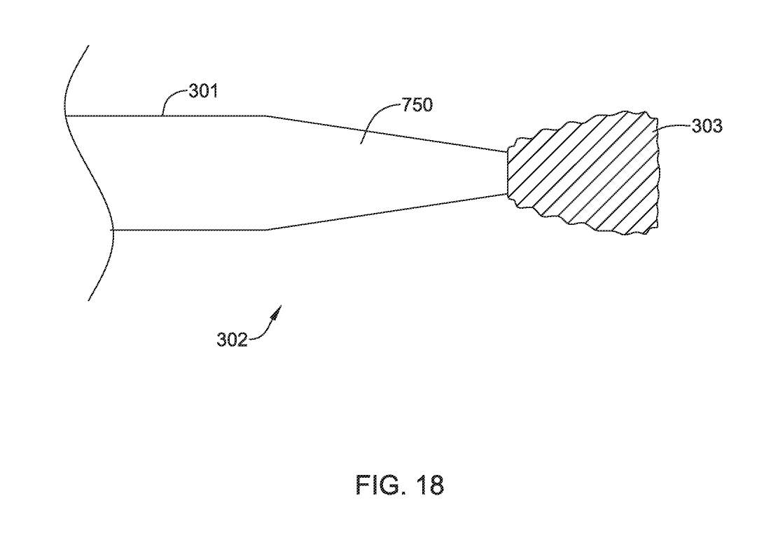

Although only described with respect to dilation device 400, various embodiments of dilation devices 200 and 300 may also include a rigid tip portion. As a few examples, FIGS. 17 and 18 depict distal portions 202 and 302 of dilation devices 200 and 300 including a rigid tip portion. In the example of FIG. 17, rigid tip portion 650 is shown connected to balloon member 203 and extending distally away from balloon member 203 of dilation device 200. Although, in embodiments where balloon member is an annular balloon disposed around the end of elongate member 201, rigid tip portion 650 may be attached to the distal-most end of elongate member 201.

FIG. 18 depicts distal portion 302 of dilation device 300, including rigid tip portion 750. As shown, rigid tip portion 750 is shown disposed between elongate member 301 and balloon member 303. For instance, rigid tip portion 750 may be connected to elongate member 301, and balloon member 303 may be connected to rigid tip portion 750. In such embodiments, rigid tip member 750 may additionally define a lumen, and the lumen defined by rigid tip member 750 may be in communication with the lumen defined by elongate member 301 and with the interior of balloon member 303. Accordingly, when inflation media is delivered into the lumen defined by elongate member 301, the inflation media may flow through rigid tip portion 750 and into balloon member 303.

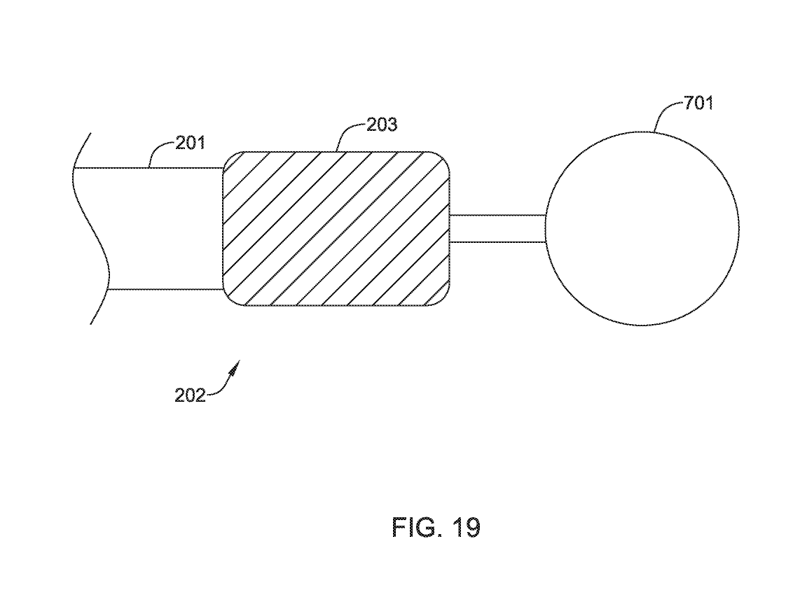

In some additional or alternative embodiments, dilation devices 200, 300, and/or 400 may further include a swallowing member, for example as depicted in FIG. 19. FIG. 19 depicts distal portion 202 of dilation device 200, including swallow member 701. As described above, balloon member 203 may be an annular balloon disposed around a distal portion of elongate member 201, and elongate member 201 may define a guidewire lumen extending through balloon member 203. In such embodiments, swallow member 701 may be attached to the distal end of elongate member 201. In some of these embodiments, guidewire port 205 (not shown in FIG. 19) may be additionally extend through swallow member 701. Although, in other embodiments, swallow member 701 may extend distally of guidewire port 205. In some embodiments, swallow member 701 may have a generally round shape, although in other embodiments swallow member 701 may have other shapes. For instance, swallow member 701 may be shaped similar to a pill. In at least some embodiments, swallow member 701 may have a diameter larger than balloon member 203 and/or elongate member 201. Although, in other embodiments, this may not be the case.

Generally, swallow member 701 may be configured in shape or size to aid patient 10 in swallowing dilation device 200. Accordingly, swallow member 701 may be a solid object. For instance, swallow member 701 may be comprised of a biocompatible metal. However, in other embodiments, swallow member 701 may be malleable. In at least some examples, swallow member 701 may be a semi-solid material. Accordingly, in some instances swallow member 701 may have a dynamic viscosity of between 150 Pascal seconds and 300 Pascal seconds, and in some particular examples may have a dynamic viscosity of 250 Pascal seconds. Dynamic viscosities in these ranges may allow patient 10 to more easily swallow swallow member 701 while still allowing swallow member 701 to pass through stricture 14. Swallow member 701 may additionally have a significant weight relative to the rest of elongate member 201. This weight may assist advancement of elongate member 201 through esophagus 12, for example due to the peristaltic contractions of esophagus 12. In different embodiments, swallow member 701 may weigh between about one ounce (28.3 grams) to about five ounces (141.7 grams), or about 2 to 100 grams, or about 2 to 5 grams, or between about 2 and 25 grams, or between about 25 grams and about 100 grams.

In some examples, to advance dilation device 200 into esophagus 12, the patient may place elongate member 201 in their mouth. In these examples where dilation device 200 includes swallow member 701, the patient may then swallow member 701, which provides rigidity and weight to dilation device 200 around which the swallowing action of the patient may more easily take. Additional swallowing and/or normal peristaltic motion of esophagus 12 may help to advance elongate member 402 into esophagus 12.

Although FIG. 19 only shows swallow member 701 in conjunction with dilation device 200, at least some embodiments of dilation devices 300 and 400 may also include swallow members. In such embodiments, the swallow members may be attached to the distal end of dilation devices 300 and/or 400. In some particular embodiments of dilation device 400, instead of being disposed attached to the distal end of a swallow member may be disposed within the lumen defined by distal elongate member 403.

In still additional or alternative embodiments, some embodiments of dilation devices 200, 300, and or 400 may further include a magnetic sensor disposed proximate the distal end 202, 302, and/or 402. For instance, a physician or other practitioner may have previously placed a magnetized body within patient 10 proximate the location of stricture 14. Where dilation devices 200, 300, and or 400 include a magnetic sensor, when the magnetic sensor is disposed in close proximity to the magnetized body, the magnetic sensor may send a signal to a notification device attached to proximal end 204, 304, and/or 404 of dilation devices 200, 300, and or 400. The notification device may be, for example, a light, a speaker or other electronic noisemaker, or a display. Accordingly, the notification device may notify patient 10 when the magnetic sensor is in proximity to the magnetized body. In this manner, patient 10 may be able to determine where dilation devices 200, 300, and or 400 are disposed in relation to stricture 14 before dilating stricture 14.

Those skilled in the art will recognize that aspects of the present disclosure may be manifested in a variety of forms other than the specific embodiments described and contemplated herein. Additionally, although various features may have only been described in conjunction with a particular Figure or embodiment, each feature described with respect to each embodiment may be combined with each other feature described herein in other contemplated embodiments. For instance, some features may have been only described with respect to dilation device 200. However, at least some contemplated embodiments of dilation devices 300 and/or 400 include the features exclusively detailed with respect to dilation device 200. Accordingly, departure in form and detail may be made without departing from the scope and spirit of the present disclosure as described in the appended claims.

What is claimed is:

1. A device for locating strictures, comprising:
an elongate member having a proximal end and a distal end;
a stopper attached to the distal end of the elongate member; and
distance markings disposed on the elongate member.

2. The device of claim 1, wherein the stopper comprises a balloon member.

3. The device of claim 1, where the stopper is cone shaped, and wherein the base of the cone shape is attached to the distal end of the elongate member.

4. The device of claim 1, wherein the stopper is shaped as a frustum of a cone, and wherein the base of the frustum of the cone is attached to the distal end of the elongate member.

5. The device of claim 1, wherein the stopper is umbrella shaped.

6. The device of claim 1, wherein the stopper comprises one or more arms connected together to form a ball-like shape.

7. The device of claim 1, wherein the stopper includes a living hinge.

8. The device of claim 1, wherein the elongate member further includes a frangible portion.

9. The device of claim 1, wherein the stopper contains one or more frangible portions.

10. The device of claim 1, further comprising a marker adjustably disposed on the elongate member.

11. A method of locating a stricture, the method comprising:
inserting a stricture locating device into an esophagus of a patient, wherein the stricture locating device comprises:
an elongate member having a proximal end and a distal end,
a stopper attached to the distal end of the elongate member, and
distance markings disposed on the elongate member;
pushing the stopper past the stricture; and
retracting the stricture locating device until the stopper contacts the stricture.

12. The method of claim 11, wherein the stricture locating device further comprises a marker adjustably disposed on the elongate hinge, the method further comprising:
adjusting the marker to contact the lips or teeth of the patient.

13. A dilation device for insertion into an esophagus, comprising:
an elongate member having a proximal end and a distal end, wherein the elongate member has a tip member bent at an angle relative to the elongate member disposed at the distal end of the elongate member;
a reservoir connected to the proximal end of the elongate member; and
a valve disposed between the proximal end of the elongate member and the reservoir,
wherein the elongate member defines an inflation lumen.

14. The dilation device of claim 13, further comprising a balloon member connected to the tip member, and wherein the inflation lumen opens into the balloon member.

15. The dilation device of claim 13, wherein the elongate member comprises a proximal section having a first diameter and a distal section having a second diameter, wherein the second diameter is greater than the first diameter.

16. The dilation device of claim 13, wherein the elongate member comprises a non-compliant material.

17. The dilation device of claim 13, wherein at least a portion of the elongate member has an inner diameter of between about 0.7 inches and about 1.2 inches.

18. The dilation device of claim 13, wherein at least a portion of the elongate member has an inner diameter of about 0.9 inches.

19. The dilation device of claim 13, wherein a distal portion of the elongate member is tapered.

20. The dilation device of claim 13, wherein at least a portion of the tip member is rigid.
